(19) United States
(12) Patent Application Publication
(10) Pub. No.: US 2009/0323370 A1
(43) Pub. Date: Dec. 31, 2009

(54) Intraoral Illuminating Apparatus

(76) Inventor: Cha Hyoung Koo, Seoul (KR)

Correspondence Address: THE NATH LAW GROUP, 112 South West Street, Alexandria, VA, 22314, US

(21) Appl. No.: 12/083,632

(22) PCT Filed: Sep. 20, 2006
(86) PCT No.: PCT/KR2006/003730

§ 371 Date: Apr. 20, 2009

(30) Foreign Application Priority Data
Oct. 17, 2005 (KR) 10-2005-0097624
May 10, 2006 (KR) 10-2006-0042084

(51) Int. Cl. A61B 1/24; H05B 39/00; F21L 4/00; G08B 5/00

(52) U.S. Cl. 362/573; 315/313; 362/183; 340/815.4

(57) ABSTRACT

An intraoral illumination apparatus is disclosed. The intraoral illumination apparatus of the present invention includes a pair of holders (1), a plurality of illuminants (7), which are provided in each holder (1), and a link unit (2), which connects the holders (1) to each other. Each N holder (1) has a U-shaped cross-section and is made of harmless synthetic resin. Furthermore, the outer surface of the holder may be coated with latex, rubber, silicone or the like, which is soft and harmless to humans. Several illuminants (7) are provided in each holder (1) at positions adjacent to the oral cavity. LEDs (light emitting diodes) or other various well-known illuminants may be used as the illuminants (7). The present invention can illuminate the oral cavity or larynx of a patient without casting shadows and prevent a dark unlit spot from being created in the oral cavity or larynx, thus facilitating medial examination and treatment.

### TECHNICAL FIELD

The present invention relates, in general, to medical illumination apparatuses and, more particularly, to an intraoral illumination apparatus which illuminates the oral cavity or larynx of a patient.

### BACKGROUND ART

Generally, illumination apparatuses, which illuminate the oral cavity or larynx of a patient for dental or otorhinolaryngological examination or treatment, have been proposed and used. Typically, such an illumination apparatus is mounted to a dental chair.

However, the conventional illumination apparatus may cast a shadow of a medial professional or an examination tool while illuminating the oral cavity or larynx of the patient. Furthermore, there is a disadvantage in that a dark spot where light cannot be radiated may occur in the oral cavity.

### DISCLOSURE OF INVENTION

### Technical Problem

Accordingly, the present invention has been made keeping in mind the above problems occurring in the prior art, and an object of the present invention is to provide an intraoral illumination apparatus which can illuminate the oral cavity or larynx of a patient without creating a shadow, and which can prevent a dark spot from occurring in the oral cavity or larynx, thus facilitating medial examination or treatment.

### Technical Solution

In order to accomplish the above object, an intraoral illumination apparatus according to a first embodiment of the present invention includes a pair of holders, each having at least one illuminant; and a link unit, having a pair of links coupled to the respective holders to couple the holders to each other. The links are spreadable, using both an adjustment shaft provided at predetermined positions through the links and an adjustment nut screwed to the adjustment shaft.

The intraoral illumination apparatus may further include a control box, which has an indicating device to indicate the operating condition of the illuminants and control the operation of the illuminants.

Furthermore, the holders may further have a switching unit to switch power supplied to the illuminants, and a wireless interface circuit connected to the switching unit. The wireless interface circuit may receive a wireless control signal from a remote control.

The intraoral illumination apparatus may further include an extension provided on each of the pair of holders so as to be movable inside and outside the holder; and an illuminant mounting member provided on an end of the extension, with at least one illuminant provided in the illuminant mounting member.

An intraoral illumination apparatus according to a second embodiment of the present invention includes a pair of holders, each having at least one illuminant; and a link coupling the pair of holders to each other, the link being spreadable in opposite directions and elastically returnable.

An intraoral illumination apparatus according to a third embodiment of the present invention includes an illumination unit, including a pair of holders, each having at least one illuminant, a pair of links coupled to respective holders, and a body, in which a battery is removably received; and a charging unit to charge the battery of the illumination unit. The illumination unit is removably mounted to the charging unit.

The body may couple the pair of links to each other and may have a plurality of charging terminals, at predetermined positions spaced apart from each other on a lower surface thereof, and an on/off switch which controls on/off operation of the illuminants.

The charging unit has: a frame, including a plurality of slots, each of which receives the illumination unit and has a plurality of charging terminals that electrically contact the respective charging terminals of the body; a cover openably coupled to the frame; a sensor to detect whether the cover is open or closed; a control unit to detect a charging state of the illumination unit and determine whether the cover is open or closed; and a display unit to display the charging state of the illumination unit using a signal transmitted from the control unit.

Preferably, the intraoral illumination apparatus according to the third embodiment of the present invention may further include a power supply cable removably coupled to the frame; and a curing lighter connected to the frame.

Furthermore, the intraoral illumination apparatus may further include a sterilizing unit provided in one of the frame and the cover such that the sterilizing unit is adjacent to the illumination unit.

In addition, the sterilizing unit comprises one selected from among an ultraviolet LED, an ultraviolet lamp and an ozone generating lamp.

### ADVANTAGEOUS EFFECTS

As describe above, the present invention can illuminate the oral cavity or larynx of a patient without casting shadows, and can prevent a dark spot in illumination from occurring in the oral cavity or larynx, thus facilitating medial examination or treatment.

### BRIEF DESCRIPTION OF THE DRAWINGS

FIG. 1 is a schematic view of an intraoral illumination apparatus, according to a first embodiment of the present invention;

FIG. 2 is a view showing the intraoral illumination apparatus, which is mounted to opposite ends of the lips of a patient to illuminate the oral cavity, according to the first embodiment of the present invention;

FIGS. 3 and 4 are views showing an intraoral illumination apparatus, according to a second embodiment of the present invention;

FIG. 5 is a view showing the intraoral illumination apparatus of FIG. 1, which is mounted to the upper and lower lips of a patient to illuminate the oral cavity;

FIG. 6 is a view of an intraoral illumination apparatus, according to a third embodiment of the present invention;

FIG. 7 is a view of an intraoral illumination apparatus, according to a fourth embodiment of the present invention;

FIG. 8 is a view showing an example of use of the intraoral illumination apparatus of FIG. 7;

FIG. 9 is a perspective view of an intraoral illumination apparatus, according to a fifth embodiment of the present invention;

FIG. 10 is a perspective view showing an illumination unit according to the fifth embodiment of the present invention;

FIG. 11 is a bottom perspective view showing the illumination unit of the fifth embodiment of the present invention;

FIG. 12 is a view showing a body according to the fifth embodiment of the present invention;

FIG. 13 is a block diagram showing a battery according to the fifth embodiment of the present invention;

FIG. 14 is a perspective view showing a charging unit according to the fifth embodiment of the present invention;

FIG. 15 is a block diagram showing the charging unit and a sterilization unit according to the fifth embodiment of the present invention;

FIGS. 16 and 17 are views showing the use of the intraoral illumination apparatus according to the fifth embodiment of the present invention;

FIG. 18 is a block diagram showing a charging unit and a sterilization unit according to a sixth embodiment of the present invention; and

FIG. 19 is a perspective view showing the charging unit according to the sixth embodiment of the present invention.

### BEST MODE FOR CARRYING OUT THE INVENTION

Hereinafter, preferred embodiments of the present invention will be described in detail with reference to the attached drawings.

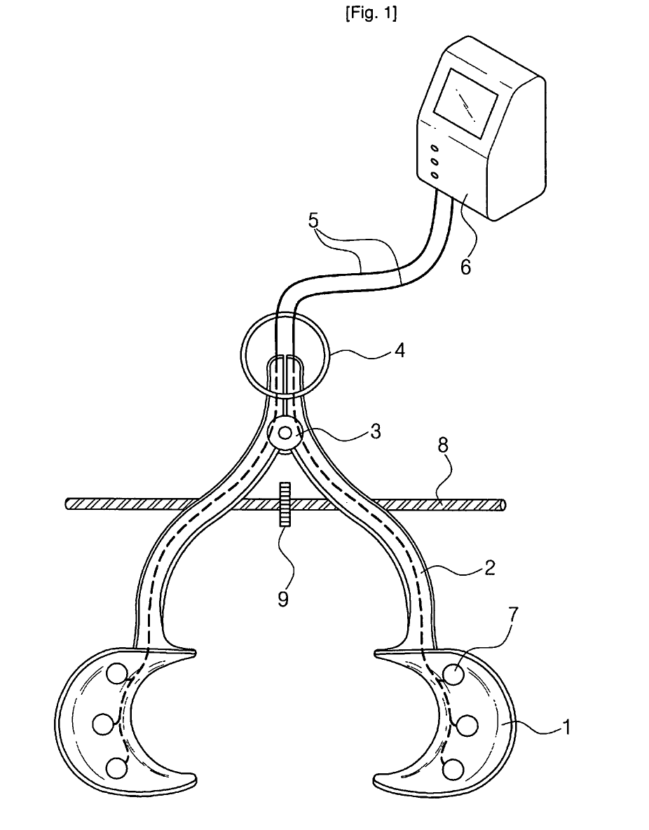

As shown in FIG. 1, an intraoral illumination apparatus according to a first embodiment of the present invention includes a pair of holders 1, a plurality of illuminants 7 provided in each holder 1, and a link unit 2, which connects the holders 1 to each other.

Each holder 1 has a U-shaped cross-section to easily wrap around the inner or outer surfaces of the lips of a patient and is made of harmless synthetic resin. Furthermore, the outer surface of the holder may be coated with latex, rubber, silicone or the like, which is soft and harmless to humans.

The several illuminants 7 are provided in each holder 1 at positions adjacent to the oral cavity. LEDs (light emitting diodes) or other various well-known illuminants may be used as the illuminants 7.

The link unit 2 includes a pair of links 2. The links 2 are coupled at first ends thereof to the respective holders 1. Second ends of the links 2 are rotatably coupled to each other by a hinge 3 and a guide unit 4 having a ring spring shape. Furthermore, an adjusting shaft 8 is provided at predetermined positions through the links 2. An adjusting nut 9 is screwed to the adjusting shaft 8. Therefore, the links 2 are rotatable around the hinge 3 and the guide unit 4, and the angle and/or distance between the links 2 can be adjusted by screw motion of the adjusting nut 9 and the adjusting shaft 8. Each link 2 may be made of material similar to that of the holder 1 or material such as aluminum, which is light and elastic.

Furthermore, power lines 5 are provided in the links 2, so that the illuminants 7 are connected to a control box 6 through the power lines 5.

The control box 6 receives power from an internal power supply or an external power supply. A display device, such as an LCD, which indicates the intensity of illumination and operating conditions, and a power switch are provided in the control box 6.

As shown in FIG. 2, the illumination apparatus of FIG. 1 is set to the lips of a patient to spread the lips to the left and right, that is, in lateral directions, and simultaneously, to illuminate the oral cavity or larynx of the patient.

Alternatively, as shown in FIG. 5, the illumination apparatus of FIG. 1 may spread the lips in upward and downward directions using a method of slightly changing the shape of the holders 1 or adjusting the distance and/or angle between the links 2.

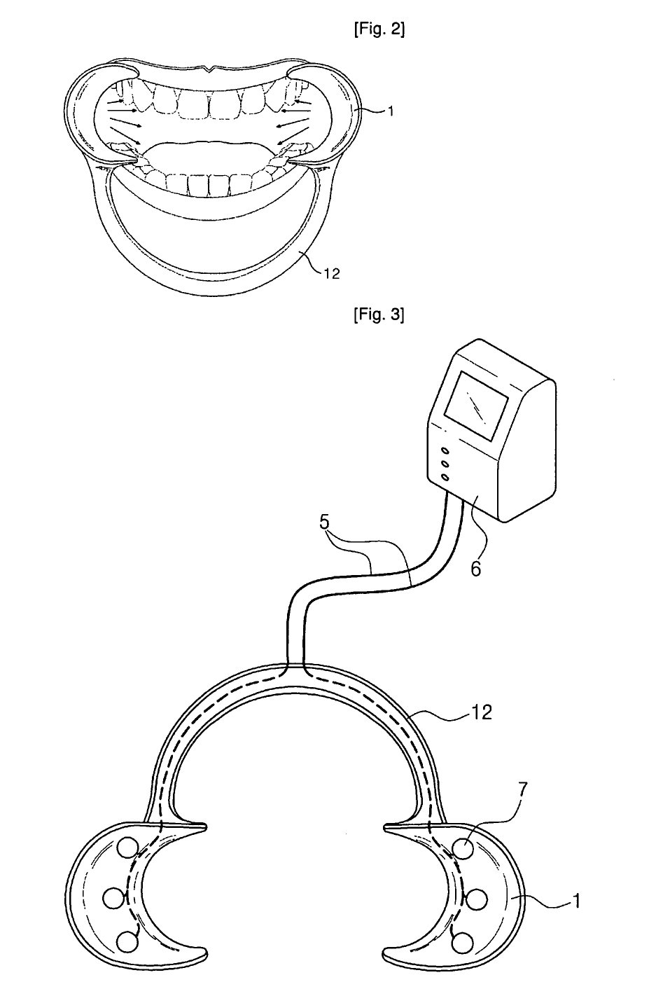

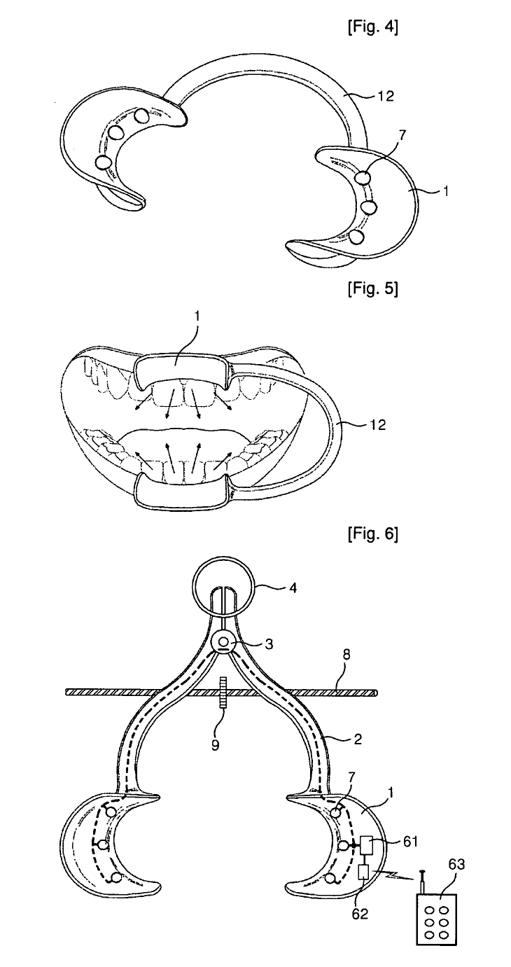

Meanwhile, as shown in FIGS. 3 and 4, in an intraoral illumination apparatus according to a second embodiment of the present invention, two holders 1 are coupled to each other through a single link 12 having elasticity. The link 12 is made of material such as synthetic resin that can have elasticity and has a U-shaped structure that can spread the mouth in lateral directions and be returned to its original state by the elasticity. The general construction and functions of the intraoral illumination apparatus according to the second embodiment, other than the above-mentioned structure, remains the same as the first embodiment, therefore further explanation is deemed unnecessary.

An intraoral illumination apparatus according to the third embodiment of the present invention shown in FIG. 6 is controlled by a wireless method, unlike the intraoral illumination apparatuses of the first and second embodiments.

As shown in the drawing, the third embodiment of the present invention includes a switch unit 61, a wireless interface circuit unit 62 and a remote control 63.

The switch unit 61 is provided in a holder 1 and includes an internal power supply, which supplies power to illuminants 7, and a switch device that controls currents, which flow between the internal power supply and the illuminants 7, using the wireless control signals of the remote control 63, which will be explained later herein.

The wireless interface circuit unit 62 is provided in the holder 1 and has an infrared or radio frequency (RF) receiving circuit. Furthermore, the wireless interface circuit unit 62 decodes a wireless control signal, which is received from the remote control 63, and transmits it to the switch unit 61.

The remote control 63 has an infrared or radio frequency transmitting circuit and transmits a wireless control signal to the wireless interface circuit unit 62.

Therefore, in the third embodiment of the present invention, operations of turning on and off the illuminants 7 and adjusting the intensity thereof can be wirelessly remote-controlled through manipulation of the remote control 63.

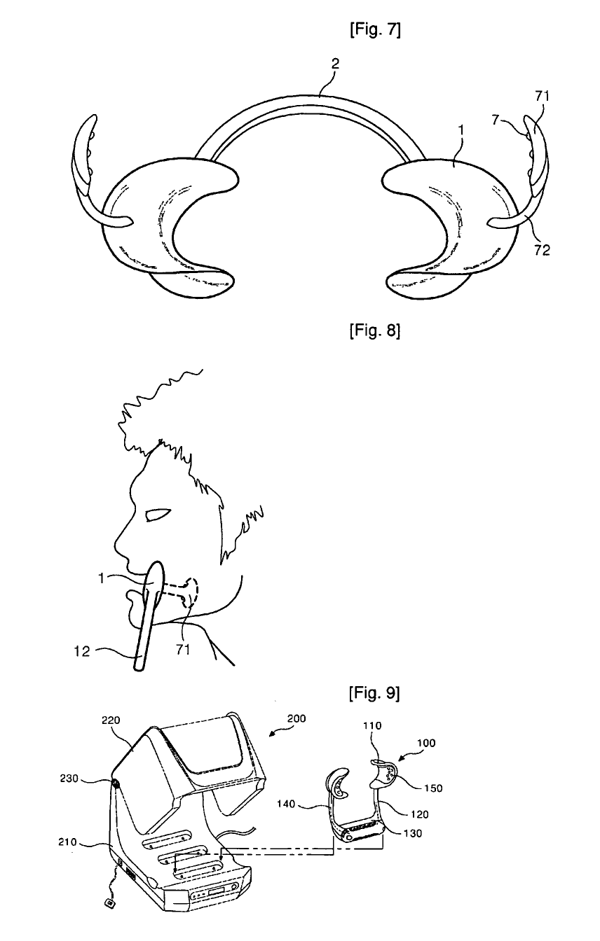

A fourth embodiment of the present invention shown in FIGS. 7 and 8 includes an extension 72, which is provided on each holder 1 so as to be movable inside and outside the holder 1, and an illuminant mounting member 71, which is provided on an end of the extension 72.

In the fourth embodiment, a plurality of illuminants 7 is provided in each illuminant mounting member 71 but not in the holder 1, and the extension 72 extends outside each holder 1. Hence, as shown in FIG. 8, the illuminants 7 can be disposed in the oral cavity at deeper positions compared to the above-mentioned embodiments, thus having an advantage in that it can illuminate the oral cavity more brightly. The general construction of the intraoral illumination apparatus according to this embodiment, other than the above-mentioned structure, is similar to that of the prior embodiments, therefore further explanation is deemed unnecessary.

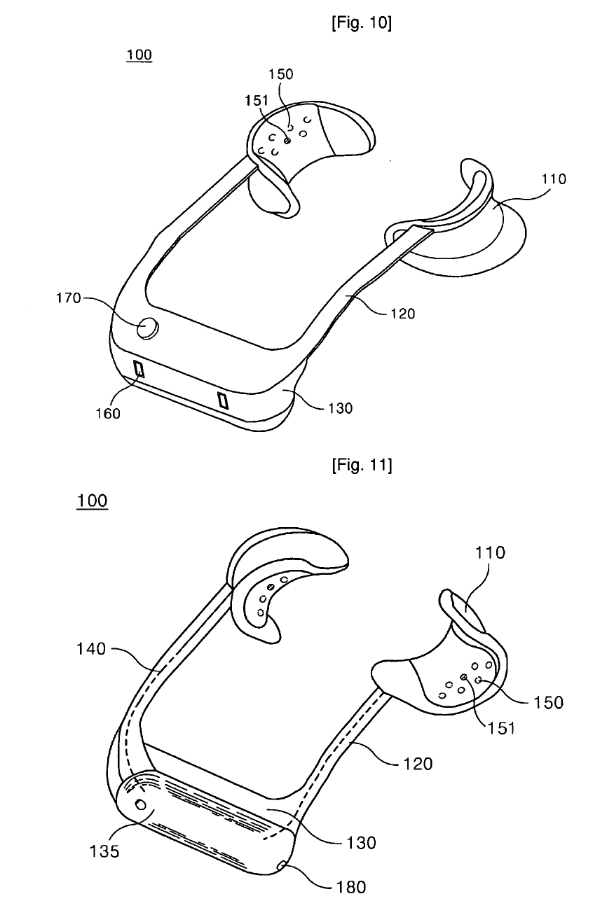

FIGS. 9 through 17 show an intraoral illumination apparatus according to a fifth embodiment of the present invention.

As shown in the drawings, the fifth embodiment of the present invention includes an illumination unit 100, which is used to spread the lips of the patient in lateral directions and illuminate the oral cavity and/or larynx, and a charging unit 200, which charges the illumination unit 100.

The illumination unit 100 includes a pair of holders 110, each of which has a plurality of illuminants 150, a pair of links 120, which are coupled to the respective holders 110, and a body 130, in which a battery 190 is removably received.

Referring to FIG. 10, each holder 110 has a U-shaped cross-section to easily wrap around the inner or outer surface of the lips of the patient, and is made of harmless synthetic resin. Furthermore, the outer surface of the holder may be coated with latex, rubber, silicone, or the like, which is soft and harmless to humans.

The several illuminants 150 are provided in each holder 110 at positions adjacent to the oral cavity. In addition, a sterilizing illuminant 151 (for example, an ultraviolet LED) may be provided at a position around the illuminants 150 in each holder 110. The ultraviolet LEDs 151 are operated along with the illuminants 150 during medical treatment or a surgical operation to protect the human body from infection by bacteria or viruses. Meanwhile, an LED or other any well-known illuminants may be used as the illuminants 7.

The links 2 and the body 130 may be made of the same material as that of the holder 1, or material such as aluminum, which is light and elastic.

Alternatively, the holders 110, the links 120 and the body 130 of the illumination unit 100 of this embodiment may be formed using resin or metal which is mixed with nano silver at an appropriate ratio. In this case, the illumination unit 100 has an antibacterial effect thanks to the nano silver.

The body 130 is coupled to the two links 120 and has at least one charging terminal 160 at a predetermined position in the lower surface thereof. An on/off switch 170, which turns on or off the illuminants 150, is provided at a predetermined position on the front surface of the body 130.

As shown in FIG. 11, a body cover 135 is coupled to the lower surface of the body 130 using a coupling member. For this, at least one first coupling hole 180 is formed through the body cover 135.

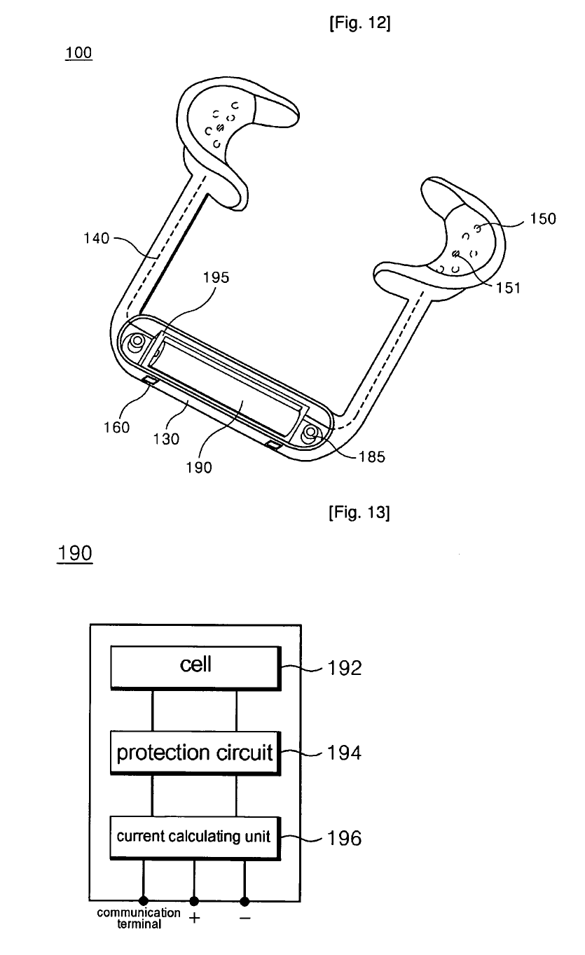

Furthermore, as shown in FIG. 12, the body 130 has therein a battery 190, which supplies power, a socket 195, which supports the battery 190 and is electrically connected to the charging terminal 160, and a second coupling hole 185, which is formed for coupling the body cover 135 to the body 130. The on/off switch 170 controls the supply of power for charging the battery 190 through the switching operation thereof. For this, power lines 140 are provided in the respective links 120, so that the illuminants 150 are electrically connected to the battery 190 through the power lines 5.

Meanwhile, as shown in FIG. 13, the battery 190 includes a cell 192, which charges and discharges power, a protection circuit 194, which protects the cell 192, and a current calculating unit 196, which calculates the amount of current supplied to the cell 192 and the amount of current discharged from the cell 192. The protection circuit 194 prevents the cell 192 from being overcharged and/or completely discharged by overcurrent or overvoltage. The current calculating unit 196 calculates the amount of current charged into and discharged from the cell 192 through power input and output terminals (+, −) and transmits the calculated amount of current to a control unit (not shown) of a main body 210 through a communication terminal. The control unit displays the charging state of the battery, which is placed in the main body 210, through a display unit.

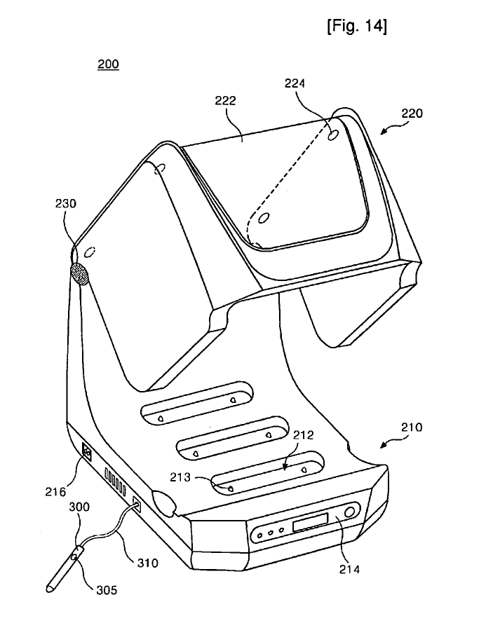

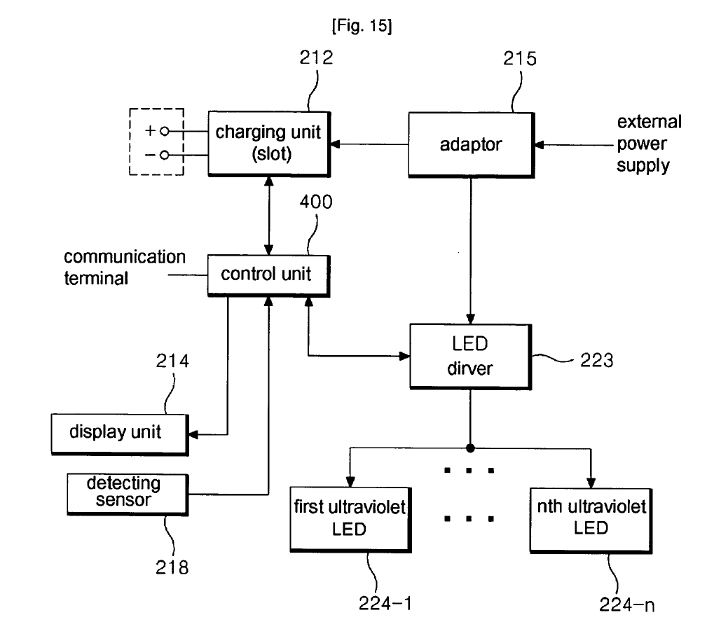

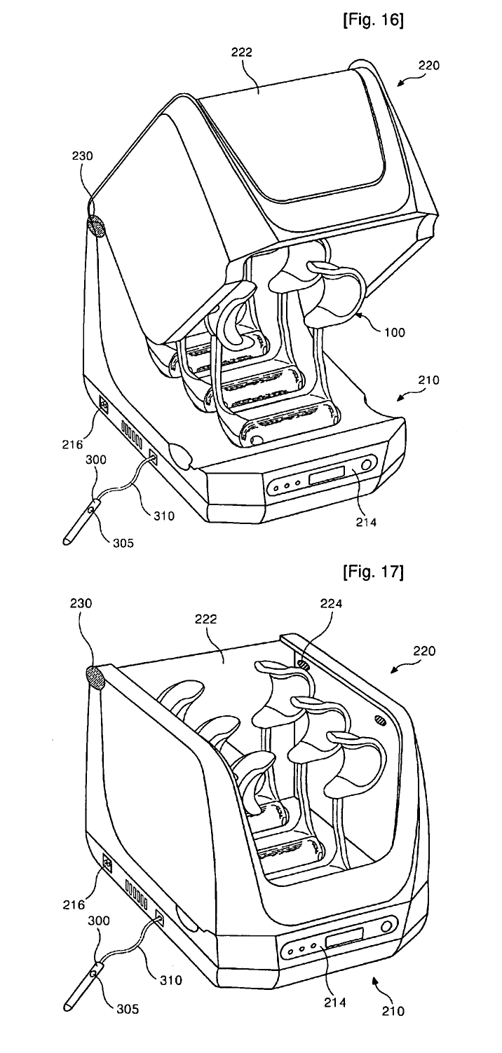

FIG. 14 is a perspective view of the charging unit. FIG. 15 is a block diagram of the charging unit.

Referring to FIG. 14, the charging unit 200 includes a frame 210, which charges the battery 190 of the illumination unit 100, and a cover 220, which openably covers the opening of the frame 210. The cover 220 is rotatably coupled to the frame 210 using hinge parts 230.

The frame 210 has a plurality of slots 212, each of which receives the illumination unit 100 to charge it, a display unit 214, which displays information about charging and/or sterilization, and a power supply unit 216, which receives power from an external power supply.

A spring type charging terminal 213, which is brought into contact with the charging terminal 160 of the illumination unit 100, protrudes into each slot 212 such that power supplied from the power supply unit 216 can be charged into the illumination unit 100. The display unit 214 is provided at a lower position on the front surface of the frame 210 to display information about charging and/or sterilization under control of the control unit.

Furthermore, a curing lighter 300, which is used as a radiation device for radiation-curing dental restorative composite resin, may be coupled to the frame 210 through a power cable 310 that extends from a predetermined position of a lower end of a sidewall of the frame 210. A power on/off switch 305 is provided at a predetermined position in the curing lighter 300. The operation of the curing lighter 300 is controlled by manipulation of the switch. Typically, the curing lighter 300 has an LED as a light source. This LED receives direct current power from the charging unit 200 and generates light having wavelengths ranging from 420 nm to 480 nm. As such, in the case where the curing lighter 300 is operated through the power cable 310, it can cure an object for a long time while maintaining a high and constant intensity of light, compared to a typical charging type curing lighter. Therefore, the time required for curing treatment, which is 30 seconds in the conventional art, can be reduced to 3 seconds or less.

The cover 220 is open at the lower and rear surfaces thereof, and other surfaces thereof are closed. The cover 220 openably closes the frame 210, and an internal space is defined by the frame 210 and the cover 220 when the cover 220 is closed to the frame 210. The illumination units 100 are received in the internal space. Furthermore, a transparent window 222 is applied to almost the entire area of the front and upper surfaces of the cover 220, such that, even when the cover 220 is closed, a user can observe the received illumination units 100. Particularly, a plurality of sterilizing units 224, which serve to sterilize the illumination units 100, is provided on the inside of the upper surface of the cover 220, which faces the received illumination units 100. When the cover 220 is closed, the sterilizing units 224 may be simultaneously automatically operated or manually operated. The power of the frame 210 and the sterilizing units 224 of the cover 220 are electrically connected to each other through electric wires, which are not shown in the drawings.

Furthermore, for example, an ultraviolet ray generator, such as an LED or a cold cathode fluorescent lamp, which generates ultraviolet rays to sterilize an object, or an ozone generator, which generates ozone to sterilize an object, may be used as each sterilizing unit 224. Here, in the case where the cold cathode fluorescent lamp is used, a separate power supply device (inverter) is required, but is not shown in the drawings. In the case where the ozone generator is used, an air intake chamber and an air discharge chamber, air circulation holes, which ensure a path for air mixed with ozone, a discharge hole, which discharges air outside after a sterilization process has been completed, and an air intake blower fan are required, but these are not shown in the drawings either. Alternatively, an ozone generating lamp may be used as the ozone generator.

Meanwhile, in the case where the ultraviolet LED is used as the sterilizing unit 224, as shown in FIG. 15, it is preferable that a plurality of ultraviolet LEDs 224-1 through 224-n be provided. As shown in FIG. 14, the ultraviolet LEDs 224-1 through 224-n may be provided on the inner surfaces of the sidewalls of the cover 220 at positions adjacent to the received illumination units 100.

Hereinafter, processes of charging and sterilizing the illumination units using the sterilizing units 224 will be described with reference to FIG. 15. When the illumination units 100 are loaded into the respective slots 212 of the frame 210, direct current power, which has passed through an adaptor 215 provided in the frame 210, is applied to the batteries 190, which are received in the respective illumination units 100, through the power input/output terminals (+, −), thus charging the batteries 190. The current calculating units 196 of the batteries 190 calculate the amount of current supplied to or discharged from the cells through the input/output terminals (+, −) and transmit the calculated amount of current to the control unit 400 through the communication terminals. The display unit 214 displays information about the charging conditions using control signals of the control unit 400. The communication terminals transmit the calculated value to the control unit (not shown) of the frame 210 so that the control unit 400 displays the charging and discharging state of the batteries, received in the frame 210, using the display unit 214. Such a process of charging the batteries can be conducted even before the cover 220 is closed, as shown in FIG. 16, and can also be conducted even after the cover 220 is closed, as shown in FIG. 17.

Meanwhile, to sterilize the illumination units 100, which are in states of being charged or have been completely charged, as shown in FIG. 8, the user must close the cover 220. After the cover 220 has been closed, the user pushes a sterilizing operation button, which is disposed at a predetermined position in the display unit 214. Then, the ultraviolet LEDs 224-1 through 224-n are operated by an LED driver 223, which is operated using direct current power transmitted from the adaptor 215, thus starting the process of sterilizing the illumination units 100. At this time, in the display unit 214, an LED lamp for indicating the sterilization process is turned on under the control of the control unit 400. Here, the user can set the time required for sterilization using input buttons. After the set time for sterilization has passed, the ultraviolet LEDs 224-1 through 224-n are automatically turned off, and the sterilization indicating LED is also turned off. Furthermore, even while the ultraviolet LEDs 224-1 through 224-n are operated, if the cover 220 is opened or a sterilization stop button is pushed, the control unit 400 stops the operation of the LED driver 223 to stop the sterilization process, and sends a signal to the display unit 214 such that the sterilization indicating LED is turned off.

The operation of sterilizing the ultraviolet LEDs 224-1 through 224-n is conducted independently of whether the batteries of the illumination units 100 are charged. Furthermore, the operation of charging the batteries of the illumination units 100 is also conducted independently of the sterilizing operation. In addition, if the cover 220 is not completely closed, even if a sterilization function input button is pushed, the LED driver 223 is not operated, due to the control of the control unit 400, which receives a detection signal from a detecting sensor 218. To indicate this to the user, the control unit 400 controls the sterilization indicating LED lamp such that it flashes on and off.

After the charging and sterilizing processes have been normally completed, the control unit detects this and applies control signals to the charge circuit and the LED driver such that they are turned off. Furthermore, the control unit 400 also sends the display unit to turn on a charge completion LED lamp and a sterilization completion LED lamp. Thereafter, when the user opens the cover and removes the illumination units, the control unit detects this and sends a signal to the display unit to turn off the two above-mentioned LED lamps.

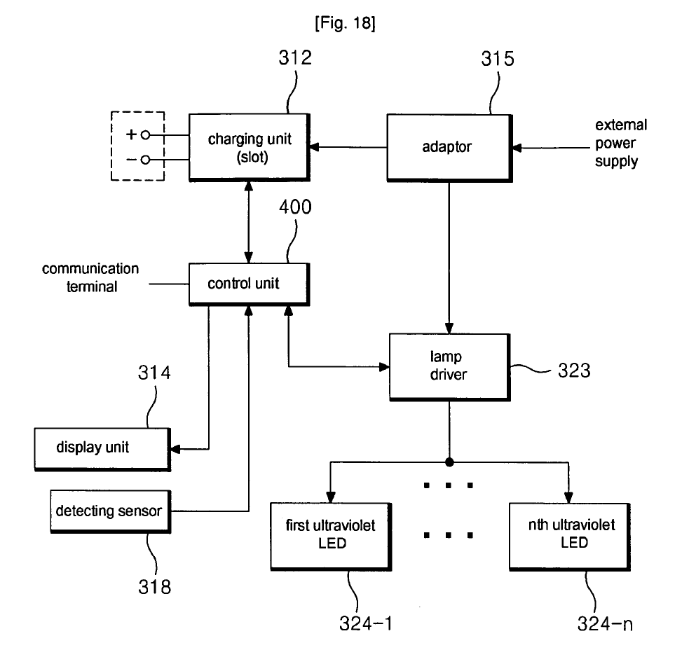

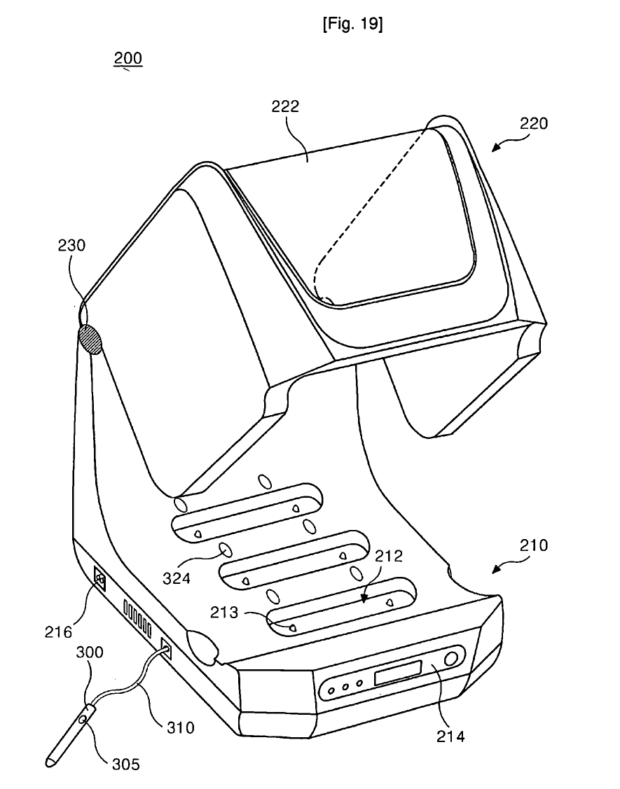

FIGS. 18 and 19 are views showing a charging unit and a sterilizing unit of an intraoral illumination apparatus according to a sixth embodiment of the present invention.

As shown in the drawings, the sterilizing unit 324 of the sixth embodiment of the present invention includes a plurality of ultraviolet lamps 324-1 through 324-n. The ultraviolet lamps 324-1 through 324-n are operated by a lamp driver 323, which is operated using direct current power transmitted from an adaptor 315, thus sterilizing the illumination units 100. The general construction of the intraoral illumination apparatus according to this embodiment, other than the above-mentioned structure, is the same as the fifth embodiment, therefore further explanation is deemed unnecessary.

Furthermore, the ultraviolet LED 224-1 through 224-n and the ultraviolet lamps 324-1 through 324-n of the sterilizing units 224 and 324 may be provided on the inner surfaces of the sidewalls of the cover 220, as shown in FIGS. 14 and 17, or, alternatively, may be provided between the slots 212 of the frame 210, as shown in FIG. 19.

Although the preferred embodiments of the present invention have been disclosed for illustrative purposes, those skilled in the art will appreciate that various modifications, additions and substitutions are possible, without departing from the scope and spirit of the invention as disclosed in the accompanying claims. Therefore, the technical bounds of the present invention are not limited to the above-mentioned embodiments but are defined by the claims.

### INDUSTRIAL APPLICABILITY

As described above, the present invention provides an intraoral illumination apparatus which is mounted to the lips of a patient to spread the lips in opposite directions and, simultaneously, to illuminate the oral cavity or larynx. Furthermore, the intraoral illumination apparatus of the present invention prevents a dark spot from occurring in the oral cavity or larynx, thus facilitating medical examination and treatment.

1. An intraoral illumination apparatus, comprising:
a pair of holders, each having at least one illuminant; and
a link unit having a pair of links coupled to respective holders to couple the holders to each other, the links being spreadable using both an adjustment shaft provided at predetermined positions through the links and an adjustment nut screwed to the adjustment shaft.

2. The intraoral illumination apparatus according to claim 1, further comprising:
a control box including an indicating device to indicate an operating condition of the illuminants and controlling operation of the illuminants.

3. The intraoral illumination apparatus according to claim 1, wherein the pair of holders further have a switching unit to switch power supplied to the illuminant, and a wireless interface circuit connected to the switching unit, wherein the wireless interface circuit receives a wireless control signal from a remote control.

4. The intraoral illumination apparatus according to claim 1, further comprising:
an extension provided on each of the pair of holders so as to be movable inside and outside the holder; and
an illuminant mounting member provided on an end of the extension, with at least one illuminant provided in the illuminant mounting member.

5. An intraoral illumination apparatus, comprising:
a pair of holders, each having at least one illuminant; and
a link coupling the pair of holders to each other, the link being spreadable in opposite directions and elastically returnable.

6. An intraoral illumination apparatus, comprising:
an illumination unit, including a pair of holders, each having at least one illuminant, a pair of links coupled to respective holders, and a body, in which a battery is removably received; and
a charging unit to charge the battery of the illumination unit, wherein
the illumination unit is removably mounted to the charging unit.

7. The intraoral illumination apparatus according to claim 6, wherein the body couples the pair of links to each other and has a plurality of charging terminals at predetermined positions spaced apart from each other in a lower surface thereof, and an on/off switch which controls on/off operation of the illuminants.

8. The intraoral illumination apparatus according to claim 6, wherein the charging unit comprises:
a frame, including a plurality of slots, each of which receives the illumination unit and has a plurality of charging terminals that electrically contact the respective charging terminals of the body;
a cover openably coupled to the frame;
a sensor to detect whether the cover is open or closed;
a control unit to detect a charging state of the illumination unit and determine whether the cover is open or closed; and
a display unit to display the charging state of the illumination unit using a signal transmitted from the control unit.

9. The intraoral illumination apparatus according to claim 8, further comprising:
a power supply cable removably coupled to the frame; and
a curing lighter connected to the frame.

10. The intraoral illumination apparatus according to claim 8, further comprising:
a sterilizing unit provided in one of the frame and the cover such that the sterilizing unit is adjacent to the illumination unit.

11. The intraoral illumination apparatus according to claim 10, wherein the sterilizing unit comprises one among an ultraviolet LED, an ultraviolet lamp and an ozone generating lamp.
